(19) United States
(12) Patent Application Publication
(10) Pub. No.: US 2019/0126751 A1
(43) Pub. Date: May 2, 2019

(54) Built-In Breathalyzer for a Vehicle

(71) Applicant: Arjun Vinay Joshi, Mountain View, CA (US)

(72) Inventor: Arjun Vinay Joshi, Mountain View, CA (US)

(21) Appl. No.: 15/798,396

(22) Filed: Oct. 31, 2017

(51) Int. Cl. B60K 28/06; G01N 33/497

(52) U.S. Cl. CPC B60K 28/06; G01N 33/4972

(57) ABSTRACT

A factory installed breathalyzer for a vehicle is disclosed. The breathalyzer can be installed in one of many spaces or cavities of the vehicle including the steering wheel assembly, the central console and the ceiling. The breathalyzer is hard wired to the vehicle's computer and is managed by the vehicle's computer.

### CROSS-REFERENCE TO RELATED APPLICATIONS

This application claims the benefit of U.S. Provisional Application Ser. No. 62/414,293, filed Oct. 28, 2016, and entitled “BUILT-IN BREATHALYZER FOR A VEHICLE,” the entirety of which is expressly incorporated herein by reference.

### TECHNICAL FIELD

The subject disclosure relates generally to breathalyzers.

### BACKGROUND

Drinking and driving is a large problem in our society. A person operating a motor vehicle while under the influence of alcohol is a danger to the health and safety of himself/herself, fellow passengers and other drivers and pedestrians on the road. For example, there is an unacceptably high chance that a person driving a car under the influence of alcohol on a highway will lose control of the car and cause a fatal accident. Thus, it is important to prevent people from driving under the influence. To that end, breathalyzers are used, for example, at parties to determine whether or not a guest at the party can safely drive himself/herself home. The breathalyzers available in the marketplace today are relatively small and portable. They are battery operated and some can be attached to smart phones and managed with smart phone's software application programs. Some can also be powered by using one of the 12 volt power outlets in a vehicle. A problem with portable breathalyzers is that they may not always be available when needed or desired, for example in public places. People have to remember to carry them in their pockets or handbags etc. Another problem is that people may not be aware that someone in their vicinity has a breathalyzer. There is no easy way to know where the nearest breathalyzer is of if a stranger is carrying one. Also, some people may not be comfortable with using a breathalyzer borrowed from another person, for example they may be fearful of ingesting another person's germs, especially if it was used by other persons recently. To reduce drinking and driving, it is desirable that breathalyzers are commonly available and their locations are well known to everyone. It is also desirable that a person's breath analysis can be done privately and without embarrassing that person in the presence of others.

### BRIEF DESCRIPTION OF THE DRAWINGS

Various non-limiting embodiments are further described with reference to the accompanying drawings in which:

FIG. 1 illustrates an example, non-limiting breathalyzer built-into the steering wheel assembly of a vehicle, in accordance with one or more embodiments described herein;

FIG. 2 illustrates an example, non-limiting tube of a breathalyzer secured to a swivel, in accordance with one or more embodiments described herein;

FIG. 3 illustrates an example, non-limiting steering column for housing harnesses or wires of a breathalyzer, in accordance with one or more embodiments described herein;

FIG. 4 illustrates an example, non-limiting breathalyzer built-into the ceiling of a vehicle, in accordance with one or more embodiments described herein;

FIG. 5 illustrates an example, non-limiting network environment in which the built-in breathalyzer can function, in accordance with one or more embodiments described herein;

FIG. 6 illustrates an example, non-limiting method for operation of the built-in breathalyzer, in accordance with one or more embodiments described herein;

FIG. 7 illustrates another example, non-limiting method for operation of the built-in breathalyzer, in accordance with one or more embodiments described herein; and

FIG. 8 illustrates an example, non-limiting computing environment in which the breathalyzer can function, in accordance with one or more embodiments described herein.

### DETAILED DESCRIPTION

One or more embodiments are now described more fully hereinafter with reference to the accompanying drawings in which example embodiments are shown. In the following description, for purposes of explanation, numerous specific details are set forth in order to provide a thorough understanding of the various embodiments. However, the various embodiments can be practiced without these specific details (and without applying to any particular networked environment or standard).

The present invention relates to implementing one or more built in breathalyzers in an automobile. In one embodiment, the breathalyzer is implemented in the steering wheel. In another embodiment, the breathalyzer is implemented in the ceiling of the vehicle, above the driver, for example in a specially created cavity inside, behind or near the sun visor. In another embodiment, the breathalyzer is implemented in the seat belt. In still another embodiment, the breathalyzer is implemented in a specially created seat cavity. In yet another embodiment, the breathalyzer is implemented in the glove compartment. In yet another embodiment, the breathalyzer is implemented inside the central console between the seats. In yet another embodiment, the breathalyzer is implemented in the dashboard. In some embodiments, special cavities, compartments or casings are built into the vehicle for housing the breathalyzer. The locations, shapes and colors of these breathalyzer housings can be selected based on aesthetic requirements, convenience of use and customer's preference.

The breathalyzer includes a mouthpiece/inlet/cavity and the person whose breathe is to be analyzed blows or exhales into it. In some embodiments, the inlet can include a tube (e.g. akin to a straw or a tube of a life vest on an airplane). In some embodiments, the tube can include an extendable tube. The tube is designed to fit into a user's mouth and can be of various shapes including round, oval or rectangular. In some embodiments, the inlet of the breathalyzer is a gas sensor that analyzes the gas in its vicinity. The gas sensor is designed to detect alcohol found in commercial alcoholic beverages, for example, ethanol. In these embodiments the user is not required to blow or exhale into the inlet. Rather, this sensor can be useful in circumstances in which the alcohol content of the user's breath is so high that the gas sensor can detect the presence of alcohol even without the user having to blow or exhale into an inlet. The gas sensor can also be configured to detect carbon mono oxide or smoke and thus serve multiple functions. The gas sensor can be located in the ceiling, steering wheel, dashboard or seat belt of the vehicle. The gas sensor can be implemented in addition to or instead of the breathalyzer.

An advantage of implementing the breathalyzer in a car as a built in standard feature is that the general public will always know that a breathalyzer is available in a nearby car. Another advantage is that a person can use the breathalyzer in the privacy of his/her car. In many instances a person will learn about his/her high alcohol content just as he/she is about to drive and make the wise determination of taking a taxi or waiting to drive until he/she is sober. Commercial breathalyzers that are pocket sized have only recently become popular. They are often less reliable than the bulkier law enforcement grade breathalyzers. A car provides an excellent environment for implementing a built in breathalyzer because it has relatively unlimited space, electrical power and computing power for implementation of sensors, in comparison to a smart phone (for example), and it is also eliminates the portability issues associated with the bulkier police grade breathalyzers. Thus, a built in breathalyzer in a car can provide the best of both worlds: portability of the pocket sized breathalyzer and reliability of the law enforcement grade breathalyzer. Also, importantly, pocket sized breathalyzers are required to be designed to use low power, be lightweight and have small form factors. There are no such severe limitations on the built in car breathalyzers, and that would allow rapid innovation in the future in the quality, functionality and features of the built in car breathalyzers.

There are also challenges associated with implementing breathalyzers and alcohol sensors in a car. One challenge is creative placement of the breathalyzer such that it is both readily accessible and also out of the way such that it does not interfere with operation of the motor vehicle. Also, the breathalyzer and its casing must be aesthetically pleasing. Another challenge is that the place of the breathalyzer must be selected such that it provides for efficient connectivity of the breathalyzer to the vehicle's electrical, display, computing and communications circuitry. The present invention provides innovative solutions to those challenges. Also, the inventor believes that the relative cost of a breathalyzer is very small in comparison with the cost of a car and in comparison to the social good that the breathalyzer in a car would do. The inventor believes that cars of the future should provide built in breathalyzers as a standard feature, just like they presently provide built in air bags and cigarette lighters.

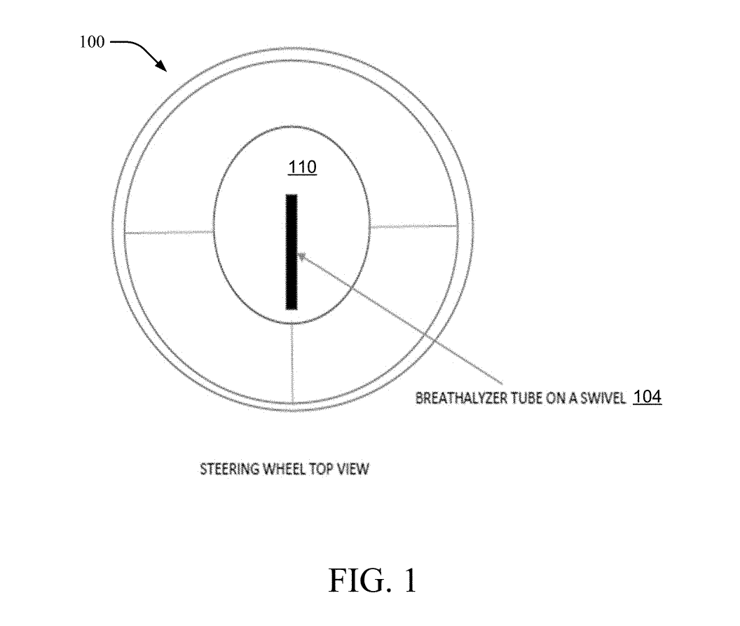

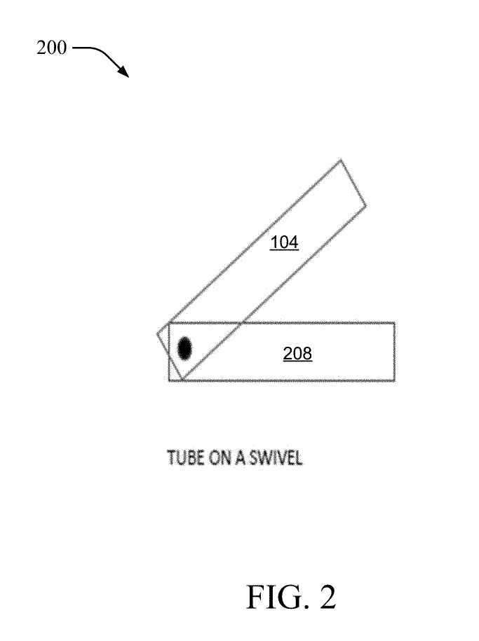

Embodiments of the present invention provide novel techniques, locations and devices for implementing breathalyzers in automobiles. FIG. 1 illustrates a built-in breathalyzer implemented in the steering wheel assembly 100 of a vehicle. The inlet tube 104 of the breathalyzer is visible to the driver and can be rotated up and down because it is connected to a swivel or a hinge. The tube can also be connected to a ball joint such that it can be rotated in three dimensions. The tube can have a mouthpiece attached to it, which can be disposable and replaceable. The driver can lift up the tube when he/she desires to use the breathalyzer. When not in use, the tube can be pushed/rotated back down into the steering wheel such that it does not distract from or interfere in the operation of the vehicle. An example implementation 200 is illustrated in FIG. 2, where the tube 104 is secured to an anchor 208 by way of a swivel. The tube 104 rotates around the swivel.

The tube can be made extendable so that the driver will have to bend less. The air exhaled by the user will have less distance to travel to the analysis chamber when the tube is not extended as compared to when the tube is extended. For some breathalyzer designs, using the extended tubing will provide a less precise measurement of the alcohol content of the user's breathe, but nonetheless a useful one, than using the non-extended tubing. So, depending on whether the user's is interested in a casual measurement or a precise measurement, he/she would decide whether or not to extend the tubing. In one embodiment, the breathalyzer includes a professional grade alcohol tester, for example, a fuel cell tester or a spectrophotometer tester. In another embodiment, the breathalyzer includes a personal grade tester for example, semiconductor oxide based tester.

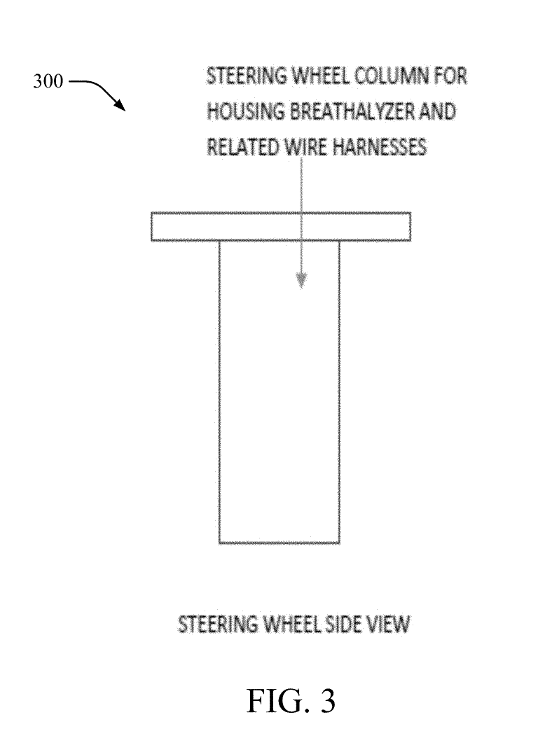

As shown in FIG. 1, the breathalyzer is housed in the steering wheel assembly 100. Specifically, the tube 104 is shown secured to, and lying flatly on top of, the steering column 110. Presently, airbags, horn and horn circuitry, wire harnesses and other control circuitry are housed in the steering column 110 underneath the steering wheel. According to an embodiment of the present invention, the breathalyzer is similarly housed in the steering wheel 110. As illustrated in FIG. 3, the steering column 300 can house the power and communication cables of the built-in breathalyzer.

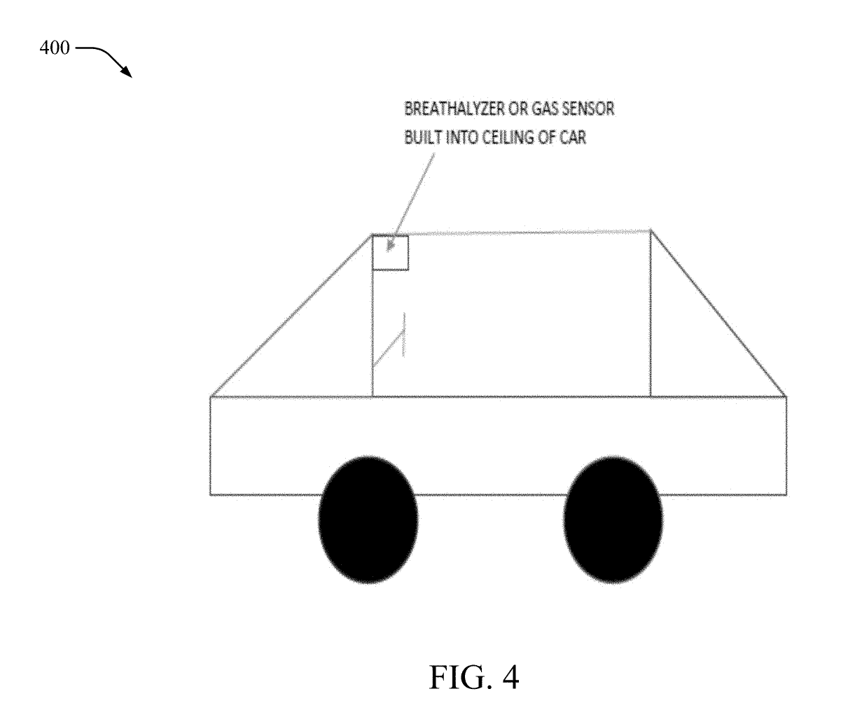

In one embodiment, only the inlet of the breathalyzer 104 is visible to the driver. In another embodiment, the inlet 104 is hidden under a cover that the driver has to lift up to access the inlet 104. In another embodiment, the breathalyzer's power and communication cables are flexible, extendable and retractable such that the driver can remove the breathalyzer from its steering wheel casing, use it and then replace it back into the casing. In one embodiment, the breathalyzer's results can be viewed on the dashboard or navigation display of the car. In one embodiment, the breathalyzer's results can be communicated (e.g. to a smart phone) by way of Bluetooth, Wi-Fi or cellular communication technologies. In one embodiment, the results of the breathalyzer and/or a gas sensor in the car can be communicated to a cloud. A police officer in the vicinity of a car can then obtain that information from the cloud. The officer will know whether or not there is a drunk driver in the vehicle in advance of approaching the vehicle. It will be helpful for the officer's safety if he/she knows in advance whether or not the driver is drunk. The breathalyzer can be programmed, reprogrammed and its software can be updated from the cloud. FIG. 4 illustrates an example implementation 400, in which a built-in breathalyzer or gas sensor housed in the ceiling of the car. As the gases exhaled by a person are often at higher temperatures than the air inside the car, and because hot air rises up, the ceiling area in front of the driver is a good location for collecting the driver's breath.

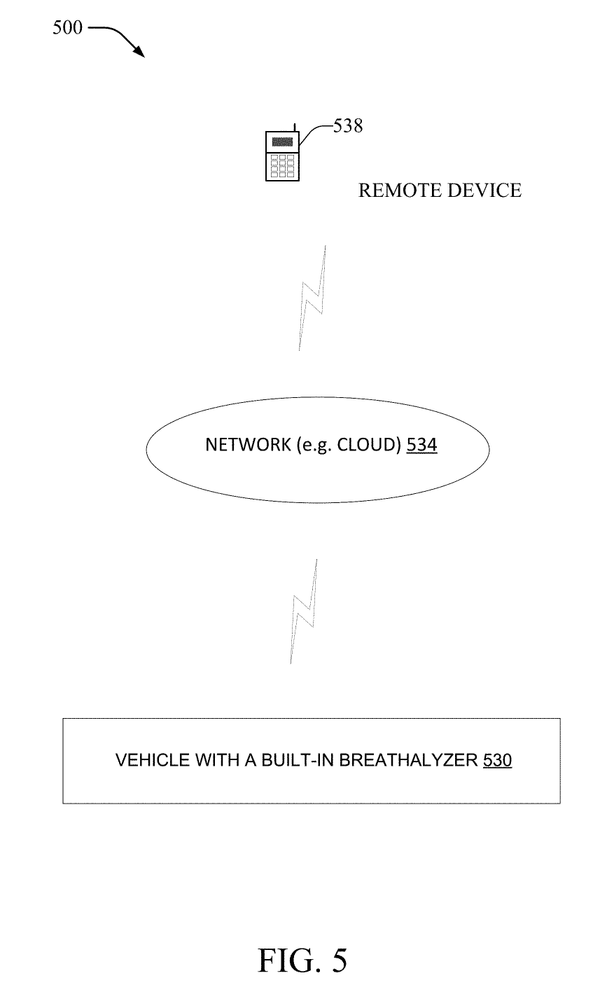

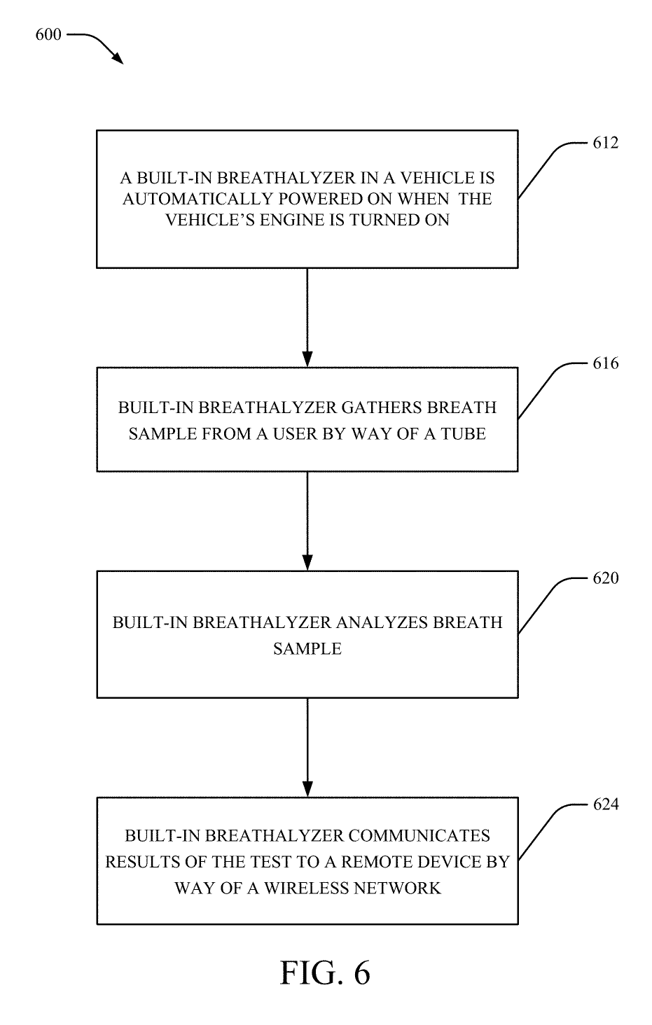

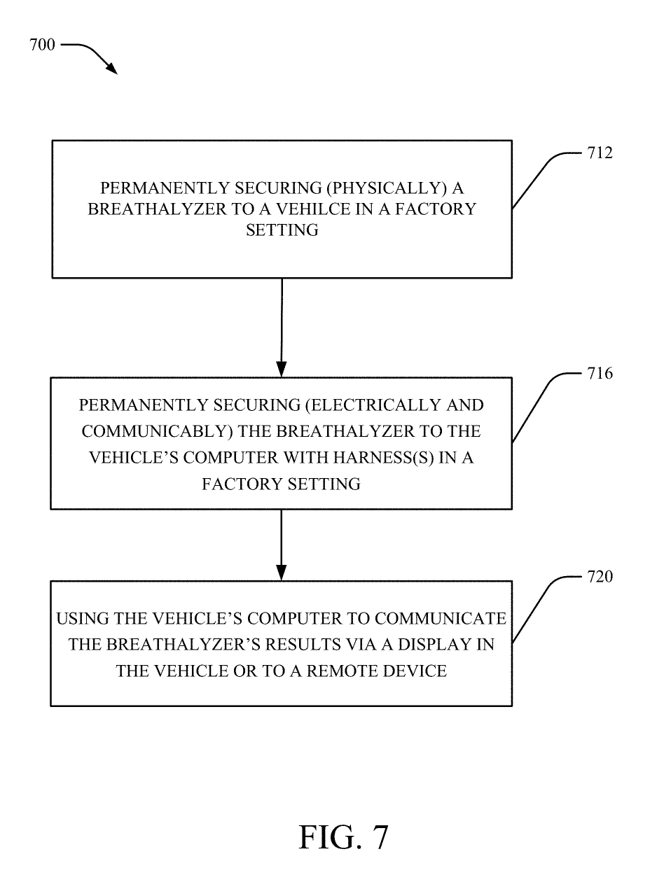

FIG. 5 illustrates an example environment 500 in which the built-in breathalyzer in a vehicle 530 communicates with a remote device 538 by way of a network 534. The remote device 538 can be a cellular phone or a database. The network can be a cloud network or a cellular network or another type of wireless network. FIG. 6 illustrates an example methodology. According to the flow diagram 600, at Step 612, a built-in breathalyzer in a vehicle is automatically powered on when a driver activates the ignition switch of the vehicle. At Step 616, the driver or user of the breathalyzer breathes into the breathalyzer's tube. At Step 620, the Breathalyzer analyzes the breath sample, either by itself or with assistance from the vehicle's computer. At Step 624, the built-in breathalyzer communicates the results of the analysis to a remote device. FIG. 7 illustrates another example methodology. According to flow diagram 700, at Step 712, a breathalyzer is permanently physically secured to a vehicle in a factory setting. At Step 716, the breathalyzer is permanently (electrically and communicably) secured to the vehicle's computer with wires or harnesses. Many, all or some of the harnesses are not visible to occupants of the vehicle. For example, because they are hidden behind panels, fabric or carpet materials. At Step 720, the vehicle's computer communicates the results of the breathalyzer, for example, on a display inside the vehicle or to a remote device.

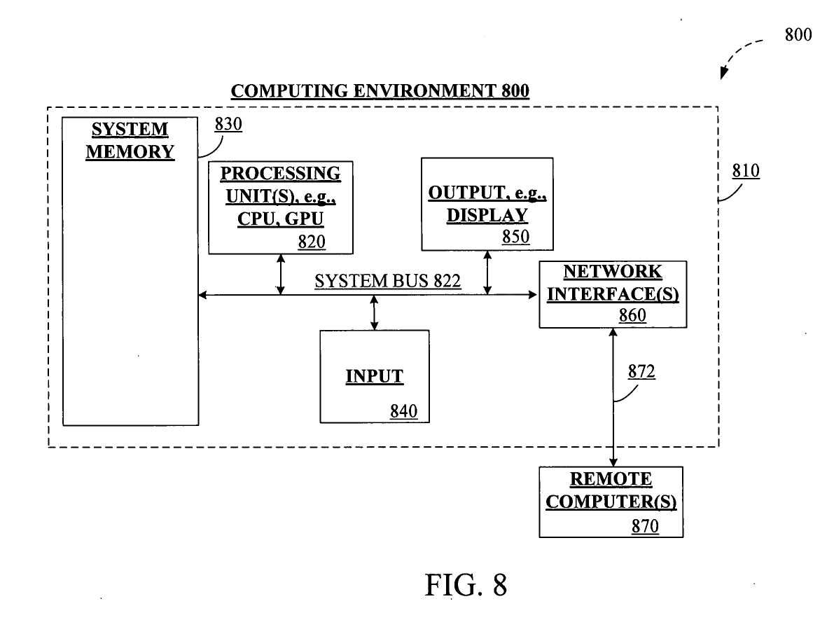

FIG. 8 illustrates an example of a suitable computing system 800 in a vehicle in which the built-in breathalyzer can function. The system 800 can include a general-purpose computing device in the form of a computer 810. Components of computer 810 can include, but are not limited to, a processing unit 820, a system memory 830, and a system bus 822 that couples various system components including the system memory to the processing unit 820.

Computer 810 typically includes a variety of computer readable media and can be any available media that can be accessed by computer 810. The system memory 830 can include computer storage media in the form of volatile and/or nonvolatile memory such as read only memory (ROM) and/or random access memory (RAM). By way of example, and not limitation, memory 830 can also include an operating system, application programs, other program modules, and program data. A user can enter commands and information into the computer 810 through input devices 810. A monitor or other type of display device is also connected to the system bus 822 via an interface, such as output interface 850. In addition to a monitor, computers can also include other peripheral output devices such as speakers, which can be connected through output interface 850.

The computer 810 can operate in a networked or distributed environment using logical connections to one or more other remote computers, such as remote computer 870. The remote computer 870 can be a personal computer, a server, a router, a network PC, a peer device or other common network node, or any other remote media consumption or transmission device, and can include any or all of the elements described above relative to the computer 810. The logical connections depicted in FIG. 8 include a network 872, such as a local area network (LAN) or a wide area network (WAN), but can also include other networks/buses.

The above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In this regard, while the subject matter has been described herein in connection with various embodiments and corresponding FIGs, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

What is claimed is:

1. A vehicle, comprising:
a factory installed breathalyzer secured to a steering wheel assembly.

2. The vehicle of claim 1, further comprising: a cavity for receiving a breath sample.

3. The vehicle of claim 1, further comprising: the breathalyzer includes a tube for receiving a breath sample.

4. The vehicle of claim 3, wherein the tube is secured to a swivel secured to the steering wheel assembly.

5. The vehicle of claim 4, wherein the tube is configured to be rotatable around the swivel.

6. The vehicle of claim 3, wherein the tube is extendable.

7. The vehicle of claim 1, further comprising: the breathalyzer is factory installed to be permanently coupled by a harness to a vehicle's computer.

8. The vehicle of claim 7, furthering comprising: the vehicle's computer is programmed to manage an operation of the breathalyzer.

9. The vehicle of claim 7, wherein the harness is situated in a steering column of the steering wheel assembly.

10. The vehicle of claim 1, further comprising: the breathalyzer is configured to communicate with a cloud network by way of a vehicle's network or a vehicle's transmitter.

11. The vehicle of claim 1, further comprising: the breathalyzer is configured to communicate with a cloud network by way of a vehicle's network or a vehicle's transmitter.

12. A vehicle, comprising:
a factory installed breathalyzer secured to a ceiling area of the vehicle.

13. The vehicle of claim 12, further comprising: a cavity for receiving a breath sample.

14. The vehicle of claim 12, further comprising: the breathalyzer includes a tube for receiving a breath sample.

15. The vehicle of claim 12, wherein the tube is secured to a swivel secured to the ceiling.

16. The vehicle of claim 14, wherein the tube is extendable.

17. The vehicle of claim 12, further comprising: the breathalyzer is factory installed to be permanently hardwired to a vehicle's computer.

18. The vehicle of claim 7, furthering comprising: the vehicle's computer is programmed to manage an operation of the breathalyzer.

19. A vehicle, comprising:
a factory installed breathalyzer secured to a central console of the vehicle.

20. The vehicle of claim 19, further comprising: the breathalyzer includes a tube for receiving a breath sample.
